(19) United States
(12) Patent Application Publication
(10) Pub. No.: US 2008/0206148 A1
(43) Pub. Date: Aug. 28, 2008

(54) Method for radiographic targeting of malignant tumors and apparatus for focusing rays

(76) Inventor: Anastasios Tousimis, Bethesda, MD (US)

Correspondence Address: OBER / KALER;A Professional Corporation, Ober, Kaler, Grimes & Shriver, 120 East Baltimore Street, Baltimore, MD, 21202-1643, US

(21) Appl. No.: 11/594,336

(22) Filed: Nov. 7, 2006

(51) Int. Cl. A61K 51/10; A61N 5/10; A61K 39/44; A61P 35/00; H05G 1/02

(52) U.S. Cl. 424/9.6; 424/9.1; 424/178.1; 600/2; 378/196

(57) ABSTRACT

A method and apparatus for immunoimaging and destruction of malignant tumors using immunoimaging agents comprising nanoclusters incorporating monoclonal antibodies that selectively bind to the cell membrane of tumor cells, and conjugated payloads comprising an encapsulated near-infrared (NIR) fluorescing crystal. The encapsulated fluorescing crystal compound provides excellent non-radiometric imaging of the tumor, and the non-antigenic metal coating encapsulating the fluorescing crystal particles provides a non-destructive necrotic killing of the tumor cells by low-level radiometric amplification and heating of the tumor cell membrane. Thus the very same nanoclusters used during imaging also serve to kill the tumor cells by necrosis, damaging the cell membrane by overheating as a result of secondary irradiation (not by harmful radiometric-induced apoptosis). Also included is a radiometric treatment platform for that better focuses the marked tumor by articulating the patient (180 degrees along one axis, 90 degrees along another), thereby minimizing incident radiation and destroying the tumor without exposing healthy tissue.

### CROSS-REFERENCE TO RELATED APPLICATIONS

The present application derives priority from U.S. provisional application No. 60/733,710 filed 4 Nov. 2006.

### BACKGROUND OF THE INVENTION

1. Field of the Invention

The present invention relates to methods and devices for more efficient imaging and destruction of malignant tumors, and especially breast cancers.

2. Description of the Background

The incidence of breast cancer, a leading cause of death in women, has been gradually increasing in the United States over the last thirty years. In 1997, it was estimated that 181,000 new cases were reported in the U.S., and that 44,000 people would die of breast cancer (Parker et al, 1997, CA Cancer J. Clin. 47:5-27; Chu et al, 1996, J. Nat. Cancer Inst. 88:1571-1579). The pathogenesis of breast cancer is largely unclear, resulting from genetic and non-genetic factors. Regardless of its origin, breast cancer morbidity and mortality increases significantly if it is not detected early in its progression. Thus, considerable effort has focused on the early detection of cellular transformation and tumor formation in breast tissue.

This is a time of explosive growth in biology, technology, and medicine. Over the past two decades, there have been remarkable advances in cellular and sub-cellular biology at the molecular level. This new understanding of the molecular basis of disease has created new demands on imaging for diagnostic and monitoring requirements. Efforts to detect and diagnose disease, to target therapies, and to monitor results are now directed at the molecular level, and biomedical imaging has become a key technology for accomplishing this. Diagnostic imaging can now characterize molecular targets and molecular processes with spatial as well as quantitative accuracy. Moreover, it is now possible to image cells and sub-cellular structures in the intact living mammal. Advances have been reported in modalities familiar to the radiologist, such as magnetic resonance imaging and ultrasound, and in emerging technologies such as optical coherence tomography (OCT). Although imaging of cellular or sub-cellular structure is important, imaging techniques that reflect physiologic processes or metabolic activity are significantly more important. For example, in the past, the usual endpoint for screening of potential cancer drugs was anti-proliferative activity. Today the emphasis is on the drug's effect on its molecular target. Non-invasive in vivo tests are needed to determine this in humans. Magnetic resonance spectroscopy, nuclear medicine and optical techniques have great potential to provide functional information.

Unfortunately, it is well-known that radiographic techniques have the potential for harm to the patients. The risk associated with obtaining a radiographic image of a patient is significant, especially over time due to the known cumulative effect of radiation exposure. While a risk/benefit analysis usually favors the use of radiographic techniques, discretion must be used to prevent harming the radiation therapy patient. Mauer, E., Biological Effects of X-ray Exposure, Am J. Chiro Med 1(3):115-118 (1988).

FIG. 1 is a chart showing the radiation effects on DNA (Chromosome, Genes) as a function of the type of radiation, and illustrating how the radiation beam fans out after passing through the cell membrane.

FIG. 2 is a diagram illustrating the complex interaction of radiation with tissue, which is exemplary of the bubble of diffracted/reflected secondary radiation formed upon tissue penetration.

It should be apparent that if a radiation beam fans outward past a tumor, penetrates through a tumor, or generates secondary radiation that extends beyond a tumor, damage to healthy tissue can easily occur. Nevertheless, most radiographic approaches to destroying cancerous tumors do just that in their effort to kill the tumor cells by apoptosis (a programmed cell death initiated by the nucleus). To do this they target DNA in the nucleus with heavy radiometric doses. This of course increases the risk of injury.

To prevent the risks associated with overexposure, a departure from classical radiation imaging and treatment is underway. These new efforts combine molecular biology, micro-electronic-micromechanical systems (MEMS and nanotechnologies) with radically modified classical radiation procedures to visualize and destroy malignancies with a minimum of side effects to the patient.

One particular approach uses imaging agents, such as labeled ligands, to add specificity to these functional imaging methods. These labeled ligands can be attached to the surface of cancer cells and facilitate their destruction with minimum or no effect on healthy cells. Tumor cell surfaces are complex, composed of proteins, carbohydrates, and other membrane-associated determinants. It is known that the epitope (surface features) can be efficiently mapped by complementary monoclonal antibodies. Specific antibodies or antigen binding fragments thereof recognize an epitope which is associated with transformation of a normal cell to a pre-cancer cell. The epitope is not present or is present in low amounts in normal cells and is highly expressed in precancer and cancer cells. Moreover, using this mechanism such antibodies can efficiently deliver payloads, and such tumor-specific antibodies are useful for targeted therapeutics that rely upon delivery to the tumor cell.

As an example, one known indicator is CD20. CD20 is a non-glycosylated 33-37 KD phosphoprotein. The expression of CD20 at high surface densities on malignant lymphoma cells has provided the rationale for the development of markers to this cell surface target. Three antibody products, Rituxan, Zevalin, and Bexxar, directed to the CD20 marker, have received FDA approval in the United States for lymphoma therapy. The use of these reagents is helping oncologists to design more effective treatment regimens.

It would be greatly advantageous to provide a novel method for immunoimaging of malignant tumors using nanoclusters that conjugate monoclonal antibodies to selectively bind to the cell membrane of tumor cells, the antibodies carrying payloads suitable for near infra-red imaging and diagnosis of human tumors and, especially breast tumors. This would eliminate much of the risk associated with radiometric imaging. The risk can be further reduced by using the same nanoclusters to kill the tumor cells, not by radiometric-induced apoptosis, but simply by generating a secondary radiation at the cell membrane to overheat the membrane, thereby providing a necrotic means for killing the cell.

FIG. 3 is a chart showing the parameters under which a cell can survive, the variables including temperature, pH, and ionic bond strength. In looking at the chart it should be apparent that the cell cannot survive at temperatures in excess of 55 degrees C., regardless of the other variables. Therefore, rather than bombarding the tumor cells with hazardous radiation, it would be greatly advantageous to kill them necrotically by overheating their membrane, causing complete cell breakdown.

### SUMMARY OF THE INVENTION

It is, therefore, an object of the present invention to combine three components into a nanocluster: 1) non-antigenic metal (such as gold); 2) antibodies; 3) fluorescent label; the three being conjugated together to provide a tool for more efficient, effective and less harmful way of imaging and destroying tumors and especially breast tumors.

It is another object to provide a method and apparatus for immunoimaging and destruction of malignant tumors using nanoclusters comprising antibodies to selectively bind to the cell membrane of tumor cells, an encapsulated fluorescing crystal compound to assist in imaging, and a non-antigenic metal coating encapsulating the fluorescing crystal particles to assist in radiometric dose amplification and destruction.

It is still another object to use the very same nanoclusters (used during imaging) to kill the tumor cells.

It is still another object to kill the tumor by necrosis, damaging the cell membrane by overheating, not by radiometric-induced apoptosis, by simply using the nanoclusters to generate a secondary radiation at the cell membrane to overheat it.

It is still another object to provide a radiometric platform for destroying the tumor that better focuses the marked tumor by articulating the patient (180 degrees along one axis, 90 degrees along another), thereby minimizing incident radiation and destroying the tumor without exposing healthy tissue.

These and other objects are accomplished by a method for immunoimaging and treatment of malignant tumors using immunoimaging agents comprising the use of a nanocluster composed of monoclonal antibodies to selectively bind to the cell membrane of tumor cells, and conjugated payloads suitable for imaging and for effecting treatment of tumors, especially breast tumors. The payload comprises a detectable fluorescing label, microencapsulated and indirectly conjugated to the antibody by a non-antigenic metal such as gold. The heavy metal gold assists in radiometric dose amplification and destruction, while the microencapsulated fluorescing compound assists in imaging. A low level radiometric method for necrotic killing of tumor cells is disclosed, as well as a radiometric patient platform and method of use that moves the patient rather than the radiometric source, thereby focusing on the marked tumor as described above and destroying it without undue exposure of healthy tissue.

The present invention may also be useful for other carcinomas such as mammary, bladder, ovarian, uterine, cervical, endometrial, squamous cell and adenosquamous carcinomas, head and neck cancers, mesodermal tumors, such as neuroblastomas and retinoblastomas; sarcomas, such as osteosarcomas and Ewing's sarcoma, melanomas, ovarian, cervical, vaginal, endometrial and vulval cancers; gastrointestinal cancer, such as, stomach, colon and esophageal cancers, urinary tract cancer, such as, bladder and kidney cancers; skin cancer; liver cancer; prostate cancer, lung cancer, breast cancer, bladder cancer, stomach, colon and esophageal cancers, liver cancer, etc.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a chart showing the radiation effects on DNA (Chromosome, Genes) as a function of the type of radiation, and illustrating how the radiation beam fans out after passing through the cell membrane.

FIG. 2 is a diagram illustrating the complex interaction of radiation with tissue, which is exemplary of the bubble of diffracted/reflected secondary radiation formed upon tissue penetration.

FIG. 3 is a chart showing the parameters under which a cell can survive, and the variables which include temperature, pH, and ionic bond strength.

FIG. 4 is a flow chart illustrating the method steps of the present invention.

FIG. 5 is a sequential schematic (to scale) of the antibody labeling procedure of the present invention.

FIG. 6 is a high-resolution SEM photograph of the nanoclusters attached on the membrane with primary antibodies visible.

FIG. 7 is an electron micrograph of an adenocarcinoma with fluorescing nanoclusters illuminated by lines 10 around the cell membranes.

FIG. 8 illustrates the process of cell death by overheating.

FIGS. 9-10 illustrate one embodiment of the patient platform for radiographic imaging and treatment according to the present invention.

FIG. 11 illustrates the striking of the nanoclusters at the cell membrane with an appropriate low-level radiation source to induce secondary radiation (bubble effect), which produces heating of the cell membrane and eventual destruction.

FIG. 12 illustrates the method of using the patient platform of FIGS. 9-10 in which the source is held stationery while the patient is rotated sufficiently to completely irradiate the entire tumor.

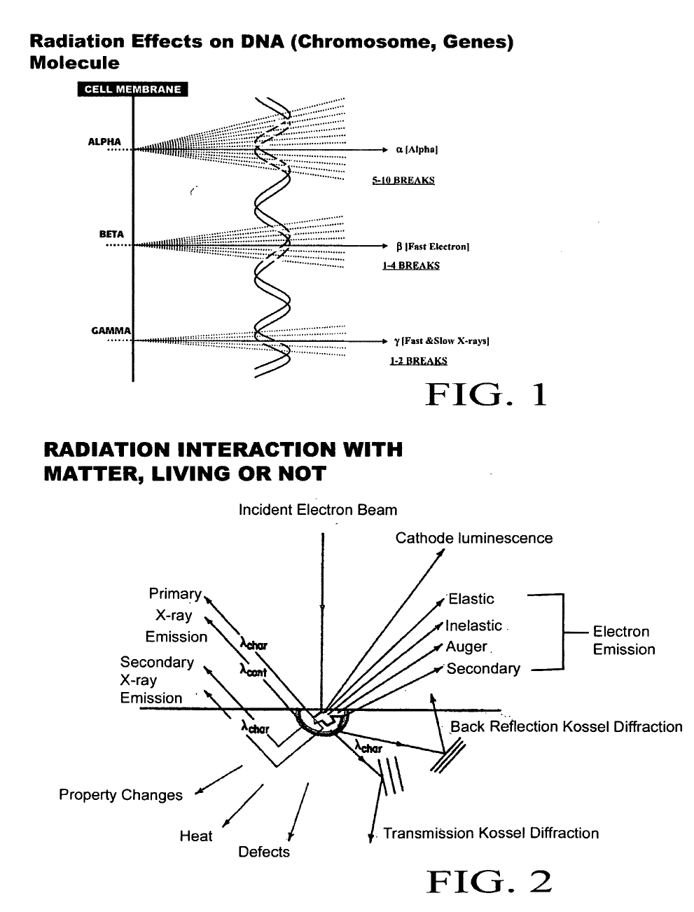

### DETAILED DESCRIPTION OF THE PREFERRED EMBODIMENTS

The present invention is a method and apparatus for immunoimaging and destruction of malignant tumors using immunoimaging agents comprising the use of nanoclusters incorporating monoclonal antibodies that selectively bind to the cell membrane of tumor cells (as opposed to penetrating the cell membrane). The antibodies carry conjugated payloads suitable for both imaging of human tumors by fluorescing, followed by destruction thereof. The novel payload comprises an encapsulated near-infrared (NIR) fluorescing crystal. The encapsulated fluorescing crystal compound provides excellent non-radiometric imaging of the tumor, and the non-antigenic metal coating encapsulating the fluorescing crystal particles provides a non-destructive platform for necrotic killing of the tumor cells by low-level radiometric amplification and heating of the tumor cell membrane. Thus the very same nanoclusters used during imaging also serve to kill the tumor cells by necrosis, damaging the cell membrane by overheating as a result of secondary irradiation (not by harmful radiometric-induced apoptosis, a conventional approach that seeks to penetrate the cell membrane with higher-level radiometric rays to directly destroy the DNA, but which inevitably destroys healthy tissue as well). The present invention also includes a radiometric platform that better focuses the marked tumor by articulating the patient (180 degrees along one axis, 90 degrees along another), thereby minimizing incident radiation and destroying the tumor without exposing healthy tissue.

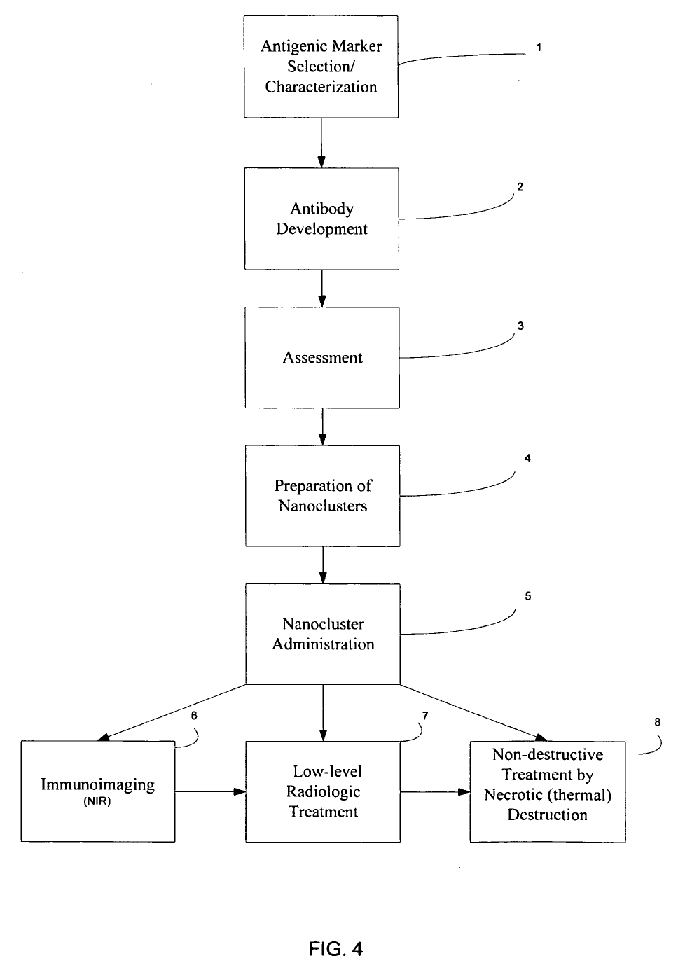

FIG. 4 is a flow chart illustrating the method steps of the present invention which will now be described in sequential manner.

### Step 1. Antigenic Marker Selection/Characterization

The preferred monoclonal antibody used for present purposes must be selective for human breast cancer outer membrane cells. Isolation of fully human affinity-matured antibodies to tumor-specific cell surface antigens has proven problematic. This initially requires the identification of an antigenic target, e.g., specific proteins or receptors expressed by the cells. Of course, antigens suitable for immunotherapeutic strategies should be highly expressed in cancer tissues and not expressed in normal adult tissues. Most breast cancer antigens discovered by serum antibody responses are intracellular, and not specific to breast tumors, and are thus of limited utility. Nevertheless, recent advances in molecular medicine have increased the interest in tumor-specific cell surface antigens that can serve as targets for various immunotherapeutic or small molecule strategies. Antigens against which a serum antibody response occurs include p53, c-myc, and c-myb. However, cell surface proteins are often poorly immunogenic for a number of reasons including shedding, glycosylation and low copy number, and so more promising antigens include Her2/neu and the B-cell antigen CD20. Other potential immunotherapeutic targets have been identified for breast cancer, including polymorphic epithelial mucin (MUC1). MUC1 is a transmembrane protein, present at the apical surface of glandular epithelial cells. It is often overexpressed in breast cancer, and typically exhibits an altered glycosylation pattern, resulting in an antigenically distinct molecule, and is in early clinical trials as a vaccine target (Gilewski et al., 2000, Clin. Cancer Res. 6:1693-1701; Scholl et al., 2000, J. Immunother. 23:570-580). The tumor-expressed protein is often cleaved into the circulation, where it is detectable as the tumor marker, CA 15-3 (Bon et al., 1997, Clin. Chem. 43:585-593). Many other genes have been reported to be overexpressed in breast cancer, such as EGFR (Sainsbury et al., 1987, Lancet 1(8547):1398-1402), c-erbB3 (Naidu et al., 1988, Br. J. Cancer 78:1385-1390), FGFR2 (Penault-Llorca et al., 1991, Int. J. Cancer 61:170-176), PKW (Preiherr et al., 2000, Anticancer Res. 20:2255-2264), MTA1 (Nawa et al., 2000, J. Cell Biochem. 79:202-212), breast cancer associated gene 1 (Kurt et al., 2000, Breast Cancer Res. Treat. 59:41-48). Other disclosures of genes and ESTs described as being expressed in breast cancer are found in international patent applications WO-99/33869, WO-97/25426, WO-97/02280 and WO-00/55173, WO-98/45328 and WO-00/22130. Similarly, genes and ESTs described as being expressed in breast cancer are disclosed in U.S. Pat. Nos. 5,759,776 and 5,693,522. All of these are potential markers for present purposes.

The proper selection of antigenic marker may depend on the particular patient characteristics. Elderly patient tumors often express different proteins and receptors than tumors arising in younger patients. Clearly, there is a range of antigenic markers that are suitably selective for detection of human breast cancer outer membrane cells, and the proper selection may be made on a case-by-case basis. For purposes of the present description, MUC1 will be used for exemplary purposes. Given the proper selection of antigenic marker, a targeting antibody can be produced.

### Step 2. Antibody Development

Given the proper antigenic marker, it is known how to target a monoclonal antibody with an affinity for human breast cancer outer membrane cells. Antibodies are generated by immunizing mice (or rabbits) with live human breast cancer cells or membrane extracts made therefrom. The mice are inoculated intraperitoneally with an immunogenic amount of the cells or extract and then boosted with similar amounts of the immunogen. Spleens are collected from the immunized mice a few days after the final boost and a cell suspension is prepared therefrom for use in the fusion. Hybridomas are prepared from the splenocytes and a murine tumor partner using the general somatic cell hybridization technique of Kohler, B. and Milstein, C., Nature (1975) 256:495-497 as modified by Buck, D. W., et al, In Vitro (1982) 18:377-381. Available murine myeloma lines, such as those from the Salk Institute, Cell Distribution Center, San Diego, Calif., USA, may be used in the hybridization. Basically, the technique involves fusing the tumor cells and splenocytes using a fusogen such as polyethylene glycol. After the fusion the cells are separated from the fusion medium and grown in a selective growth medium, such as HAT medium, to eliminate unhybridized parent cells. For the MUC1 marker, antibodies used for targeting may be synthesized from the F(ab′)2 fragments of the anti-MUC1 MAb 12H12, which reacts with the vast majority of breast tumors. A variety of known antibodies exist for other markers (see above). Humanized monclonal antibodies directed to Her2/neu (Herceptin.RTM./trastuzumab) are currently in use for the treatment of metastatic breast cancer (Ross and Fletcher, 1998, Stem Cells 16:413-428). Similarly, anti-CD20 monoclonal antibodies (Rituxin.RTM./rituximab) are used to effectively treat non-Hodgekin's lymphoma (Maloney et al., 1997, Blood 90:2188-2195; Leget and Czuczman, 1998, Curr. Opin. Oncol. 10:548-551).

Monoclonal antibodies that bind to the protein products of some of the afore-mentioned overexpressed genes have been reported (Green et al., 2000, Cancer Treat. Rev. 26:269-286), but none are currently approved for breast cancer therapy in the US. In addition, U.S. Patent Application No. 20040151724 by Julia Coronella-Wood published Aug. 5, 2004 discloses antibody fab fragments specific for breast cancer.

### Step 3. Assessment

Given a suitable antigenic marker and an antibody with affinity for the tumor cell membrane, the selectivity and range of the given antibody is determined by testing it against panels of (1) human breast cancer tumor tissues, (2) human breast cancer cell lines, and (3) normal human tissue or cells of breast or other origin. Antibodies are appropriate for breast cancer immunoimaging purposes if they have a selectivity equal to or less than 0.09 and a breast tumor binding range of equal to or greater than 0.25 or a breast cancer cell line binding range of equal to or greater than 0.25.

### Step 4. Preparation of Nanoclusters

In accordance with the present invention, the antibodies which bind to the tumor cell membrane antigen are conjugated to an infrared crystal fluorescent marker via a non-antigenic metal “linker” that may be bound to the fluorescent crystal marker and which also bind to the monoclonal antibody. Appropriate linkers will have four characteristics, three of which are well-known. First, they must be capable of binding the imaging moiety which has the desired characteristic to be read for imaging. Secondly, the linker must not significantly affect the binding selectivity of the monoclonal antibody or substantially diminish its affinity for the antigen to be bound. Lastly, the linker must form a stable bond with the fluorescent marker and the monoclonal antibody so that the fluorescent marker and antibody will not be separated from one another. In accordance with the present invention, there is a fourth requirement. The linker is a non-antigenic metal capable of being formed as an encapsulation coating around the fluorescent marker so as to additionally serve as an energy transfer medium, converting low-level radiation into thermal energy as described in detail below.

A. Linker

The preferred linker comprises any inert metal satisfying the foregoing constraints, and in the preferred embodiment comprises gold. It is known that gold particles can be bound directly to antibodies and can also serve as a binder for attachment of fluorescein thereto. Gold is known to be an excellent material for introducing surface functionality via the attachment of proteins or other macromolecules because of the metal's chemical inertness, electrical conductivity, surface uniformity and stability, biologic compatibility/low toxicity and other properties. Moreover, it is known that certain other macromolecules can then be adsorbed directly onto a clean gold surface. For example, certain classes of immunoglobulin, streptavidin, protein A and certain proteins or peptides with basic charges have been passively adsorb to gold at pH 6 to 8 in appropriate buffers containing relatively low concentrations of salts (Scopsi, et al., J. Histochem Cytochem 34:1469-1475, 1986). Gold has been used for immunocytochemistry since 1971 when Faulk and Taylor discovered adsorption of antibodies to colloidal gold. It is an ideal label for electron microscopy (EM) due to its high atomic number, which scatters electrons efficiently, and the fact that preparative methods have been developed to make uniform particles in the appropriate size range of 5 to 30 nm, 18 nm being preferred.

Possible other inert coating materials, colloidal and non-colloidal include: ceramics, such as oxides (e.g., titanium oxide, zirconium oxide, aluminum oxide, silica, glass, silicon oxynitride, iron oxide, and mixtures thereof), nitrides (e.g., titanium nitride, silicon nitride, and mixtures thereof), carbides (e.g., titanium carbide, silicon carbide, tungsten carbide, and mixtures thereof) and silicides (e.g., tungsten silicide, titanium silicide, and mixtures thereof); metals, such as copper, gold, silver, nickel, iron, cobalt, palladium, platinum, zinc, chromium, aluminum, lead, rare earth metals, and mixtures thereof; and semi-conductors, such as silicon, germanium, alloys of silicon and germanium, III-V semiconductors (e.g. gallium arsenide, aluminum phosphide, gallium nitride, and alloys thereof), and II-VI semiconductors (e.g. cadmium selenide, zinc sulfide, and alloys thereof).

Moreover, different surface protein modifications are possible that render the nanoclusters more stable under different environmental or physicochemical conditions. For example, protein shells that are inert to environmental conditions may provide for enhanced storage of the microparticle's internal contents. Such stability might preserve or otherwise extend the shelf-life of bioactive ingredients of pharmaceutical compounds. The biodegradable component of the protein shell may permit an inert microparticle to release its contents when it is needed during therapy. Nanogold™ is an existing example of a modified gold cluster comprising a gold core covalently linked to surface organic groups. These in turn may be covalently attached to antibodies. The organic groups offer better penetration into tissues, generally greater sensitivity, and higher density of labeling. This increases the range of probes possible, and expands the applications of gold labeling. Consequently, it is envisioned that the inert metal used for the present nanoclusters may include other constituents.

In the present context, fluorescing crystal particles are encapsulated in a thin film of the gold or Nanogold-like metal, and affinity-purified monoclonal antibodies are then conjugated to the microcapsules as described below. The covalent label linkage is stable indefinitely, and the attachment at a hinge thiol site ensures maximum preservation of native immunoreactivity.

B. NIR-Fluorescent Crystal Marker

As with other imaging applications, molecular imaging requires fixation of a signal-emitting molecule (e.g., one containing a radioactive, fluorescent, or paramagnetic label) within the cell or tissue where the target is expressed. The signal-emitting molecule is of prime importance in the present invention because it fluoresces upon application of near infrared light, a far less destructive approach than radiometric imaging. While there are known imaging moieties comprising infrared fluorescent dye, the preferred moiety used herein is a NIR-fluorescent nanocrystal marker. More specifically, the crystalline core comprises a BA/SR sulfur/salt doped with europeum, in powdered (nanocrystal) form. The crystals fluoresce upon application of near infrared (NIR) light, and the color (red, green, etc.) can be controlled by the doping. Injection of near-infrared nanocrystal markers permits imaging in real time using excitation fluence rates of only 5 mW/cm2, in vivo, well suited for biomedical imaging. Moreover, because the near-infrared nanocrystal markers are encapsulated in an outer coat of inert gold, this insulates the barium salt and makes it safe for injection.

C. Microencapsulation of NIR-Fluorescent Nanocrystal Markers in Gold

The NIR-fluorescent nanocrystal markers may be contained on a platform and sputter coated with the gold. This can be accomplished using an existing sputtering-gas-aggregation (SGA) source (a high-pressure magnetron-sputtering gun) using a cold sputter module in a high-vacuum evaporator equipped with an omni-rotary stage to produce monodispersed nanoclusters having very narrow size distribution. There are a variety of other established microencapsulation techniques, in addition to RF magnetron sputtering, including dual ion beam assisted deposition (DIBAD), and ion assisted dynamic mixing. The present inventor has successfully coated the NIR-fluorescent nanocrystal markers with gold using high pressure sonic vibration induced by piezoelectric speakers. All of these conventional microencapsulation techniques are considered within the scope and spirit of the present invention.

D. Conjugation to Antibodies

The above-described NIR-fluorescent nanocrystal gold nanoclusters can be conjugated to the antibodies in several ways. For one, both the antibodies and fluorescing microencapsulated fluorescing particles can be incorporated in the gold nanoparticles during the synthesis of the nanoparticles. The gold encapsulation coating may be capped with a surface layer material present in solution, such as citrate ions, thiol-containing molecules, phosphines, phenanthrolines, and silanes. Peptides containing cysteine and antibody fragments that contain a free hinge thiol group, when used as a surface layer material, can bind covalently to surface metal atoms through the sulfur atom. Lectin that is covalently attached to a phosphine, when included in the synthesis step as surface layer material, binds covalently to surface metal atoms through the phosphine group.

Alternatively, the antibodies can be attached to the gold nanoclusters after the gold nanoclusters have been produced, under conditions where the conjugates replace a pre-existing surface layer material. For example, thiol-containing molecules can replace some surface layer materials of metal nanoparticles. The kinetics of this exchange process is determined by the concentrations and binding affinities of the two binding surface materials, and heating can accelerate the process. Antibodies can displace citrate ions and become attached to the gold surface via covalent interactions (e.g., through a sulfur or phosphorus atom) and/or non-covalent interactions, such as van der Waals, hydrophobic interactions, and charge attractions. Alternatively, the antibodies can be bound to metal surfaces by multipoint non-covalent interactions, and are considered “adsorbed” to the metal particle.

Additionally, the antibodies can be linked to the gold nanoclusters in a chemical reaction through a chemically reactive group present in the conjugates and/or the surface layer material of the gold nanocluster. For example, a gold NIR-fluorescent nanocrystal that has a surface layer of phosphine where one or more of the phosphines contain a primary amine can react with a peptide that contains an N-hydroxysuccinimide ester, forming a covalent bond. A large number of cross-linking molecules are also available that enable linking of two molecules via their reactive groups.

In the preferred embodiment, the antibodies are then attached to the nanoclusters by a physico-chemical process called Organic Compound Assisted-Metal Fusion (OCAMF). Organic compounds can be adsorbed on metal colloids and cause aggregation by changing the surface zeta potentials of the particles, thereby forming stable clusters. These composite organic-inorganic nanoclusters are then used as fluorescing markers to map the tumor as well as radiologic targets for destroying the tumor by necrosis.

### Step 6. Nanocluster Administration

The organic-inorganic nanoclusters can be combined with a pharmaceutically acceptable carrier, such as solvents, dispersion media, isotonic agents and the like. Examples of carriers include water, saline solutions, sugar, gel, porous matrices, preservatives and the like, or combinations thereof.

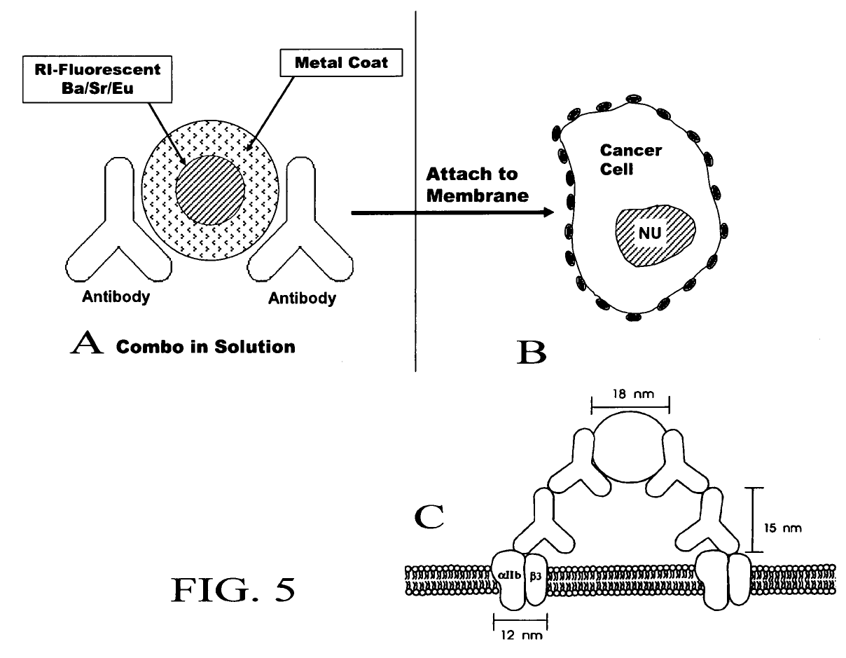

FIG. 5 is a sequential schematic (to scale) of the antibody labeling procedure of the present invention. As seen at (A), the nanocluster comprises the crystalline BA/SR sulfur/salt core doped with europeum. The core is encapsulated in a non-antigenic metal (gold) coat to form an approximately 18 nm nanocluster, having two conjugated primary antibodies. The nanoclusters are injected in solution form near the site of the tumor. The nanoclusters may be administered to a patient for imaging and/or radiation enhancement by intravenous or intra-aretrial injection, direct injection into a target tissue (e.g., tumor), and implantation of a reservoir device or cavity capable of a slow release of metal nanoparticles. Intravenous injection is well suited to delivery of metal nanoparticles to the vascular system, and is the preferred method of administration where the target tissue is a tumor. Intravenous injection permits the antibodies to directly reach their angiogenic targets at the tumor site.

Once administered, as shown at (B), the nanoclusters show an affinity for the tumor membrane surface, and as seen at (C) the primary antibodies bind the nanocluster a surface antigen on the tumor cell membrane, effectively surrounding the cell membrane. Animal experiments have shown that greater than 90% of the nanoclusters attach to the tumor membrane with very few to the liver or spleen.

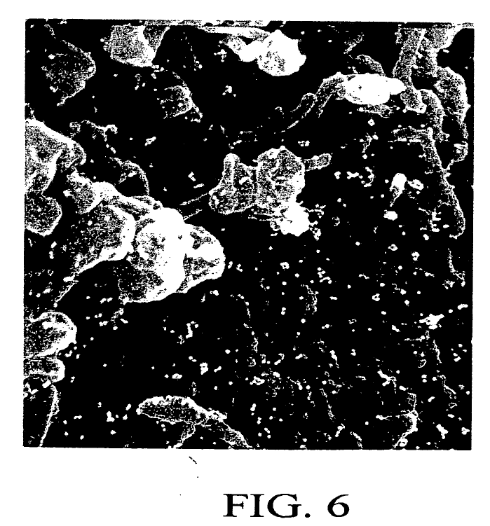

FIG. 6 is a high-resolution SEM photograph of the nanoclusters attached on the membrane with primary antibodies visible.

The nanoclusters may then be used for imaging and/or treatment as described below. The nanoclusters described above provide the vehicle for harmless near infrared imaging as well as low-level radiologic way to selectively damage or totally destroy the cancer cell membrane. Both the immunoimaging and new approach for thermal destruction of the cell membrane are described below.

### Step 7. Immunoimaging

The fluorescing nanoclusters can be used in a variety of immunoimaging procedures to detect the presence of breast cancer in a patient, monitor the status of such cancer in a patient already diagnosed to have it, and to exactly map the contours of the tumor. The NIR fluorescence may thus be utilized to: (1) determine if a tumor can be treated by an agent or combination of agents; (2) determine if a tumor is responding to treatment with an agent or combination of agents; (3) select an appropriate agent or combination of agents for treating a tumor; (4) monitor the effectiveness of an ongoing treatment; and (5) identify new treatments (either single agent or combination of agents). In particular, the identified markers may be utilized to determine appropriate therapy, to monitor clinical therapy and human trials of a drug being tested for efficacy, and to develop new agents and therapeutic combinations.

The preferred form of imaging in the present invention relies on near-infrared fluorescent imaging (fluorescent in the range of 650-800 nm) and has several advantages for in vivo imaging, including (a) high transmittance of tissue to near-infrared light as opposed to the visible light; (b) low interference of scattered light used for exciting fluorescence; and (c) nonionizing photons serve as the source of fluorescence excitation. The experimental proof of the feasibility of NIR imaging in tumors (wavelength range, 700-850 nm) using fluorescence-mediated tomography has been demonstrated by Ntziachristos V., Bremer C., Graves E. E., Weissleder R. In-vivo tomographic imaging of near-infrared fluorescent probes. Mol. Imaging, 1: 82-88, 2002, and Ntziachristos V., Tung C-H., Bremer C., Weissleder R. Fluorescence molecular tomography resolves protease activity in vivo. Nat. Med., 8: 757-760, 2002.

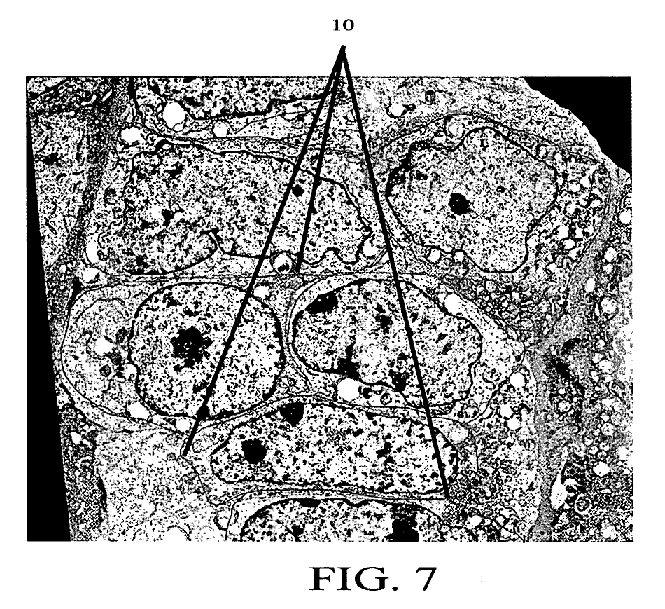

FIG. 7 is an electron micrograph of an adenocarcinoma with fluorescing nanoclusters illuminated by lines 10 around the cell membranes (these lines appear in red as depicted in a color photograph). The epitope (surface features) can be very accurately mapped by the fluorescing nanoclusters.

### Step 8. Radiologic Treatment

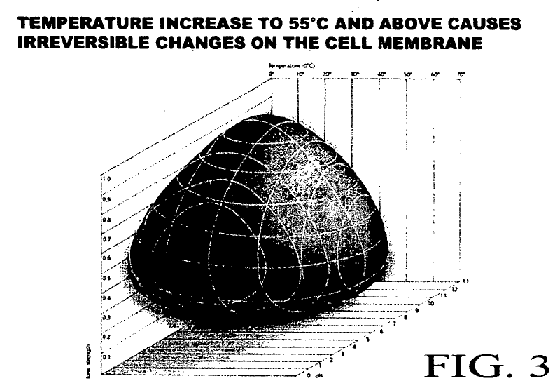

In 1926, Regaud and Lacassagne predicted that “the ideal agent for cancer therapy would consist of heavy elements capable of emitting radiations of molecular dimensions, which could be administered to the organism and selectively fixed in the protoplasm of cells one seeks to destroy”. This presumes that radiation that damages DNA stimulates cell death or apoptosis. Apoptosis, a mechanism of cell death that incurs little or no inflammatory response. Of course, effectiveness of radiation in producing cell death is dependent on dose rate as well as ionization density, and this subjects other non-tumor cells to radiation risks. These risks are here reduced in two respects, one being a more focused radiometric apparatus in which the patient is articulated (rather than the radiation source as described more fully below in section 10). The other is the low-level method of cell destruction made possible by the nanoclusters. The non-antigenic metal coating encapsulating the fluorescing crystal particles provides a non-destructive platform for necrotic killing of the tumor cells by low-level radiometric amplification and heating of the tumor cell membrane. Thus the very same nanoclusters used during imaging also serve to kill the tumor cells by necrosis, damaging the cell membrane by overheating as a result of secondary irradiation (not by harmful radiometric-induced apoptosis, a conventional approach that seeks to penetrate the cell membrane with higher-level radiometric rays to directly destroy the DNA, but which inevitably destroys healthy tissue as well). Referring back to FIG. 3, it is apparent that the cell cannot survive at temperatures in excess of 55 degrees C., regardless of the other variables. Therefore, the goal is to heat the cell membrane, killing it necrotically by overheating, causing complete cell breakdown.

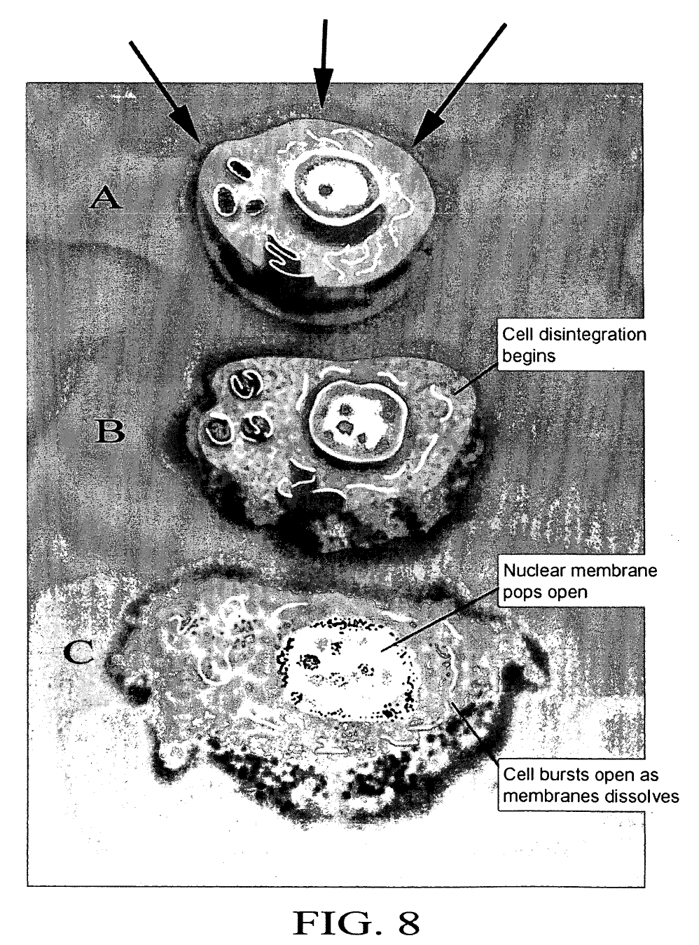

FIG. 8 illustrates this process of cell death by overheating. At (A), the cell membrane is irradiated with low-level radiation. The inherent radiation-enhancing effect of the gold-laden nanoclusters allows for dose-enhancement, and consequently reduced level of radiation for killing the tumor cells. Experimental x-ray dose enhancement adjacent to bulk metallic gold was reported by Regulla and coworkers (Regulla, D F, Hieber, L B, and Seidenbusch, M, “Physical and biological interface dose effects in tissue due to x-ray-induced release of secondary radiation from metallic gold surfaces,” Rad. Res. 150, 92 (1998)). A solid state detector was placed next to a thin (150 ÿm) gold foil and a dose enhancement factor of more than 100 with a range of 10 ÿm was found in the range of 40 to 120 kV tube potential. Cells were then placed in close proximity (2 ÿm) to the gold surface. In a clonogenic assay, 80 keV x-rays caused 80% cell killing at 0.2 Gy, which was a factor of 50 over the control without gold. Similar dose enhancements are possible with the nanoclusters of the present invention because the antibody-bound clusters essentially form a gold layer over the tumor cell membranes, thereby creating an expected dose enhancement factor of 50-100.

### Step 9. Non-Destructive Treatment

There are a variety of forms of non-destructive radiation suitable for use in practicing the methods of the present invention including, but not limited to, visible light, lasers, infrared, microwave, radio frequencies, ultraviolet radiation, and other electromagnetic radiation at various frequencies. In accordance with the present invention, these sources produce secondary effects that can be useful for destroying a target tissue through apoptosis, or more specifically, by heating the tumor cell membrane through energy absorption. Sufficient heating causes hyperthermia. Hyperthermia (or thermotherapy) is a known cancer treatment that involves heating tumor cells within the body. Elevating the temperature of tumor cells results in cell membrane damage, which, in turn, leads to the destruction of the cancer cells. Hyperthermia treatment of cancer requires directing a carefully controlled dose of heat to the cancerous tumor and surrounding body tissue. Cancerous tissues can be destroyed at exposure to a temperature of about 108° F. for an hour. Conventional hyperthermia methods have attempted to heat cancerous tumors directly, usually using microwaves to heat the high-water content of tumor cells (such tissue heats very rapidly when exposed to high-power microwaves). By concentrating the microwave energy, it is possible to selectively heat tumors and protect healthy tissues.

The present invention relies on indirect heating of the cell membrane. The nanoclusters disclosed here-above have a high affinity to the tumor cell membrane, and a high coefficient of absorption. Thus they bind to the tumor cell membrane and provide a localized mechanism for absorbing visible, ultraviolet, and infrared light particularly well. This enhances the effects of radiation absorption when present in a local vicinity that is irradiated, resulting in tissue-specific absorption. Moreover, the gold converts low-level radiation into thermal energy, thereby heating the membrane around cells of the tumor. Once the membrane achieves a temperature of 55 degrees C., as seen at (B) cell disintegration begins. This is irreversible, and so disintegration continues as seen at (C), with the cell bursting open as the membrane dissolves, and the nuclear membrane eventually popping open.

### 10. Focused Radiometric Apparatus in Which the Patient Is Articulated

Conventional radiometric treatment devices control the motion of the illumination point with respect to a stationery patient. Thus, to fully irradiate a breast tumor the illumination point must generally be moved through at least a 180 degree arc around the patient's breast. X-rays and gamma rays typically propagate outward along a cone-shaped path. Thus, for breast cancer tumors, this results in radiologic beams cutting through wide areas of healthy tissue, possibly harming the patient and inducing new cancers. An improved platform is herein disclosed in which low-level radiation is more carefully calculated so as not to penetrate through tumor cell. The low level radiation is intended to provide secondary radiation (bubble effect) which warms the cell membrane as described above, thereby killing the cell.

Since the present method is designed to irradiate the membrane only, scanning-type treatment can be made much more efficient by stabilizing the illumination point and moving the patient stage. Movement of the patient stage in three dimensions can then be used to create virtually any desired pattern of illumination.

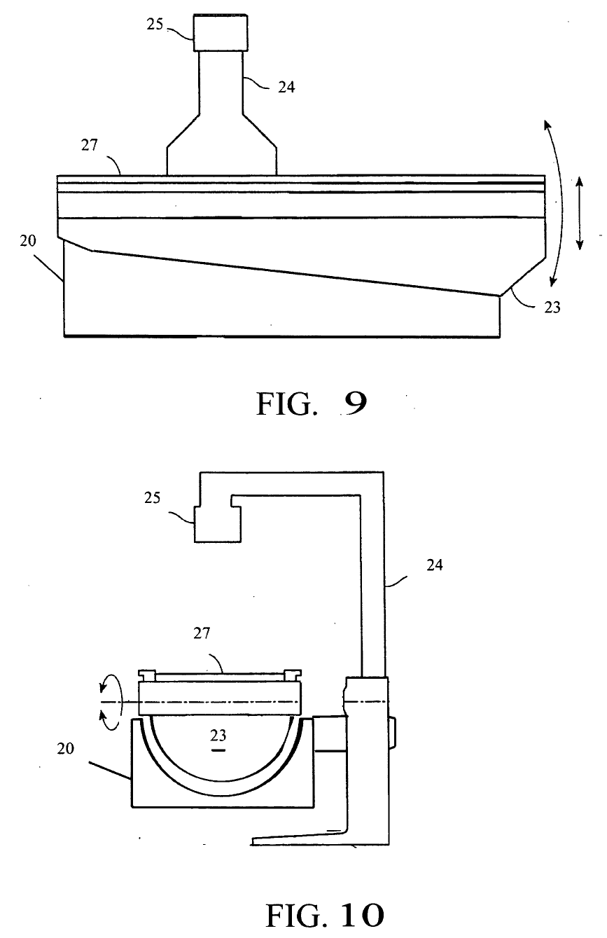

An embodiment of the apparatus for radiographic imaging and treatment according to the present invention is shown in FIGS. 9-10, and generally comprises a base 20 and a main body 23, which is supported by the base 20. The body 23 is articulated pivotally 360 degrees around a horizontal axis, and vertically 90 degrees. A supporting post 24 is attached to the base 20 for supporting a stationery radiometric source 25. A patient platform 27 is supported on the main body frame 23 between the radiometric source 25. The patient platform 27 is movable longitudinally, pivotally and vertically. In use, the tumor which is completely marked as described earlier is made the focal point of the x-ray beam, and the patient is rotated by moving the patient platform 27. Movement of the patient platform 27 in three dimensions creates virtually any desired pattern of illumination.

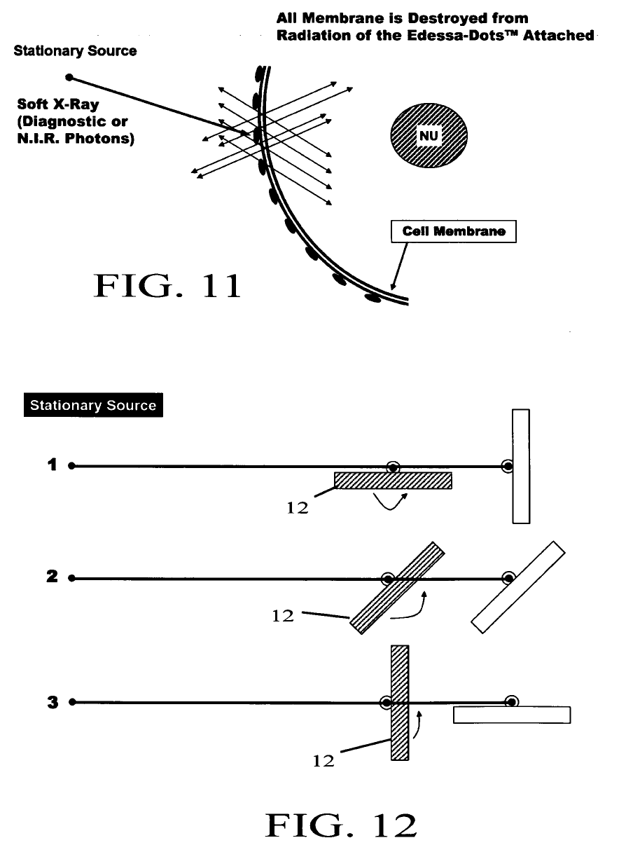

As seen in FIG. 11, the object is to strike the nanoclusters with an appropriate radiation source, whereupon secondary radiation (bubble effect) and heat produced on the membrane will destroy it. Thus, the patient is articulated to move the entire cell membrane of the tumor under the exposure of the stationery x-ray source, the patient being rotated by the patient platform 27. The stationary source of radiation may be diagnostic X-Rays, infrared photons, magnetic or any source that will cause the nanocluster antibody antigen combination to give off secondary radiation in order to heat damage the tumor cell and kill it, thus destroying the tumor. Thus, as seen in FIG. 12, the source is held stationery while the patient is rotated on the body 23 pivotally 360 degrees around a horizontal axis, and vertically 90 degrees. This is sufficient to completely irradiate the entire tumor.

It should now be apparent that the very same nanoclusters used during imaging also serve to kill the tumor cells by necrosis, damaging the cell membrane by overheating as a result of secondary irradiation (not by harmful radiometric-induced apoptosis), and thus the NIR imaging and low-level focused treatment drastically reduce incident radiation, and destroy the tumor without exposing or damaging healthy tissue.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

I claim:

1. A method for radiographic targeting of malignant tumors, comprising the steps of:
selecting an antigenic marker for said tumor membrane;
selecting an antibody having an affinity for said marker;
forming nanoclusters by microencapsulating infrared fluorescent crystals in a non-antigenic metal coating;
conjugating said nanoclusters to said antibodies to serve as a binder for attachment of said fluorescing nanoclusters to said marker;
combining said nanoclusters with a pharmaceutically acceptable carrier and administering to a patient;
imaging said nanoclusters; and
irradiating said nanoclusters with low-level radiation sufficient to induce secondary radiation and heating of said membrane in excess of 55 degrees C. to kill said tumor cells by necrosis.

2. The method of claim 1, wherein said marker is a near infrared fluorescent nanocrystal marker.

3. The method of claim 2, wherein said near infrared fluorescent nanocrystal marker comprises a crystalline core including sulfur salt doped with europium.

4. The method of claim 1, wherein said antigenic marker is polymorphic epithelial mucin (MUC1).

5. The method of claim 1, wherein said antibody having an affinity for said marker is selective for human breast cancer outer membrane cells.

6. The method of claim 1, further comprising a step of assessing selectivity and range of said antibody prior to said step of forming nanoclusters.

7. The method of claim 6, wherein said step of assessing selectivity and range of said antibody comprises testing said antibody against panels of human breast cancer tumor tissues, human breast cancer cell lines, and normal human tissue.

8. The method of claim 1, wherein said step of forming nanoclusters by microencapsulating infrared fluorescent crystals in a non-antigenic metal coating comprises the substep of using RF magnetron sputtering.

9. The method of claim 1, wherein said step of forming nanoclusters by microencapsulating infrared fluorescent crystals in a non-antigenic metal coating comprises the substep of using dual ion beam assisted deposition.

10. The method of claim 1, wherein said step of forming nanoclusters by microencapsulating infrared fluorescent crystals in a non-antigenic metal coating comprises the substep of using ion assisted dynamic mixing.

11. The method of claim 1, wherein said step of forming nanoclusters by microencapsulating infrared fluorescent crystals in a non-antigenic metal coating comprises the substep of using high pressure sonic vibration.

12. The method of claim 2, wherein said metal coating is gold.

13. The method of claim 1, wherein said step of conjugating said nanoclusters to said antibodies to serve as a binder for attachment of said fluorescing nanoclusters to said marker comprises the substep of linking said antibodies to said nanoclusters in a chemical reaction.

14. The method of claim 1, wherein said step of conjugating said nanoclusters to said antibodies to serve as a binder for attachment of said fluorescing nanoclusters to said marker comprises the substep of attaching said antibodies to said nanoclusters by Organic Compound Assisted-Metal Fusion (OCAMF).

15. The method of claim 2, wherein said imaging is accomplished with near-infrared light.

16. The method of claim 1, wherein said pharmaceutically acceptable carrier comprises any one from among a group consisting of solvents, dispersion media, and isotonic agents.

17. The method of claim 1, wherein said step of imaging said nanoclusters comprises the substep of determining if a tumor is responding to treatment.

18. An apparatus for radiographic imaging and treatment comprising:
a base;
a main body supported by said base, wherein said body is articulated pivotally 360 degrees around a horizontal axis, and vertically 90 degrees;
a supporting post attached to said base for supporting a stationery radiometric source; and
a patient platform supported on frame of said main body, wherein said patient platform is movable longitudinally, pivotally and vertically to rotate a patient such that an entire cell membrane of a tumor of said patient is under exposure of said stationery radiometric source.

19. A method for killing malignant tumors, comprising the steps of:
selecting an antigenic marker for the membrane of a tumor;
selecting an antibody having an affinity for said marker;
conjugating said antibodies to non-antigenic metal nano particles;
combining said conjugated metal nano particles with a pharmaceutically acceptable carrier and administering to a patient;
irradiating said nano particles with radiation sufficient to induce secondary heating of said tumor membrane in order to kill said tumor cells.

20. The method of claim 19, wherein said irradiating step kills said tumor cells by necrosis.

21. The method of claim 19, wherein said irradiating step induces secondary heating of said tumor membrane to at least 55 degrees C. in order to kill said tumor cells.
